(19) United States
(12) Patent Application Publication
(10) Pub. No.: US 2017/0079841 A1
(43) Pub. Date: Mar. 23, 2017

(54) DEVICE FOR A MANIPULATION WITH A CONTACT LENS WITHIN AN EYE AREA AND A METHOD OF MANUFACTURING SUCH DEVICE

(71) Applicant: Lucie PALASOVÁ, Praha 6 (CZ)

(72) Inventor: Lucie PALASOVÁ, Praha 6 (CZ)

(21) Appl. No.: 15/310,713

(22) PCT Filed: May 19, 2015
(86) PCT No.: PCT/CZ2015/000046

§ 371 Date: Nov. 11, 2016

(30) Foreign Application Priority Data
May 21, 2014 (CZ) PV2014-350

(51) Int. Cl. A61F 9/00

(52) U.S. Cl. CPC A61F 9/0061

(57) ABSTRACT

Device for a manipulation with a contact lens within an eye area and methods for the production thereof. The invention concerns a device for a manipulation with a contact lens within an eye area and methods for the production thereof, the subject matter of which lies in that it comprises a functional part (1) from porous polymer, mainly hydrophilic polymer, which is shaped for a non-aggressive contact with the cleaned area of the eye and adapted by its porousness for absorption of non-toxic and non-irritant fluid, as well as methods for producing said device.

### FIELD OF THE INVENTION

The invention relates to a device for a manipulation with a contact lens within an eye area and to a method for manufacturing thereof.

### BACKGROUND OF THE INVENTION

In case of wearing contact lenses, hygiene is the basic imperative. When introducing the contact lens into the eye or when removing it from the eye, the fingers contacting the contact lens have to be always well washed and correctly dried. Without such hand hygiene not only the contact lens are very often fouled but also an infection may be spreaded into the eye. When manipulating with the contact lens, the wearer of the contact lens removes the contact lens from its case by his finger tips. It is important to overcome a contact of the contact lens with the wearer's nails in order to prevent a damage of the contact lens. When freshly removed from its case the contact lens is soaked with a conservation solution and it is quite difficult to be manipulated when applying the contact lens on the eye, whilst being worse stickable on the eye. Thus the wearers dry the contact lens by transferring it from one finger onto another finger. It is important to have dry fingers during it as the contact lens is worse separable from wet fingers. When spontaneously drying the finger, the contact lens wearer very often uses a textile stuff to do that, however such textile can leave a yarn on the finger or possibly other dirt, thus such yarn or dirt may be transferred onto the surface of the contact lens. When any small dirt is stuck on the contact lens during its insertion, it is necessary to move the contact lens by the tummy of the index finger onto the whites and to leave the contact lens to settle back. Thus there is a chance to wash out the dirt, but even more often it is necessary to remove the contact lens from the eye by means of the fingers. When the contact lens hurts in the eye, it is necessary to remove the contact lens from the eye in any case and to wash it with a conservation liquid, inserting it again thereafter. Burning eye is very often obtained in case of an insufficient hygiene. Also numerous infections may be spreaded into the eye when manipulating with a contact lens on the eye, leading to possible inception of different eye diseases. However, burning eye can be also occur when the hands are washed with a soap being more difficult to be washed out, thus it is transferred onto the contact lens during the application. The hygiene problem during the manipulation with the contact lens starts to be critical when the hygiene imperative cannot be strictly maintained, as for example in case of staying in third world countries or when being in less developed countries in which the hygiene is basically not in such a level.

In order to prevent such above mentioned undesirable effects linked with the manual manipulation with the contact lens an applicator for a manipulation with a contact lens has been developed working on a principle of an underpressure. Said applicator is made from a resilient material such as a hollow tube that is closed on one end and it is widened on the second end to form an opened bell. The manipulation with such applicator is following. The applicator tube is pressed and the bell part of the applicator is applied on the contact lens. When the pressure on the tube is released, an underpressure in the tube is created thus sticking the contact lens to the bell part of the applicator. With such arrangement the contact lens is applied on the eye, the tube is now pressed thoroughly by the fingers so that remaining air in the tube creates an overpressure, thus separating the contact lens from the bell part of the applicator. The same process is applied in order to remove the contact lens from the eye. Even such applicator substantially eliminates the insufficient hygiene problems, there is a substantial disadvantage as the manipulation with such applicator is usable only when applying the contact lens on the eye. The use of said applicator for removing the contact lens from the eye requires an ultimate dexterity from the wearer and in many cases it requires a help from a second person.

Above mentioned insufficiencies are overcome by a device for a manipulation with the contact lens within the eye area as proposed by this invention.

### SUMMARY OF THE INVENTION

The subject of the invention is a device for a manipulation with a contact lens within the eye area, the subject matter of which resides in that it is formed by a functional part from a porous polymer, mainly hydrophilic polymer, the shape of which is adapted for a non-aggressive contact with the eye area and the porousness of which for absorption of non-toxic and non-irritant fluid, wherein the front applicator end of the functional part is provided on the surface of the functional part with at least one concave recess and/or at least one convex bulge projecting from the surface of the functional part.

Conveniently, the device includes a handle permanently or detachably connected with the functional part. Conveniently, the device is sealed in a watertight packaging. Conveniently, the device is provided with the porous polymer functional part of an average porosity 20 to 98% of the volume in relation to the total volume of the porous polymer. Conveniently, the porous polymer of the functional part has an average pore size of 0.1 to 250 micrometers. Conveniently, the porous polymer of the functional part is a deformable porous polymer.

According to another aspect of the invention, a method of manufacturing said device as defined above, is also provided, the subject of the invention residing in that the functional part is obtained from an aqueous solution of a porous polymer precursor by extrusion of the porous polymer by a removal of the water from said solution. Thus formed functional part is then eventually connected with a handle and it is eventually sealed separately or together with the handle in a watertight packaging. Advantageously, the aqueous solution of the porous polymer precursor is an aqueous solution of a polyvinyl alcohol and the porous polymer is eliminated from this solution by a separation of the water by means of repeated freezing and thawing of this solution.

The subject of the invention is also a method of manufacturing the device defined above, the subject matter of which resides in that the aqueous solution of the porous polymer precursor is an aqueous solution that contains a major part of 2-hydroxyethyl methacrylate and minor part of a multifunctional cross-linker and the extrusion of the porous polymer is carried out by a radical polymerisation in a presence of a water soluble initiator of radical polymerization. The acquired functional part is then eventually connected with a handle and separately or with the handle it is eventually sealed in a watertight packaging.

Conveniently, the expulsion of the porous polymer from the aqueous solution of the porous polymer precursor is carried out directly in the watertight packaging.

In order to make the device according to the invention multifunctional, it can be used also for removing undesirable substances and objects, such as metabolic waste products, soiling, dust and cosmetics residues from the eye area. In such case the front part of the application end of the functional part can be advantageously sharpened.

The convex bulge projecting from the surface of the application surface of functional part can be alternatively conveniently replaced by substantially convexially rounding the application end of the functional part of the device.

Conveniently, the non-toxic and non-irritant liquid is at least one fluid selected from a set comprising water, glycerol, 1,2-propylene glycol, triethyleneglycol, dimethyl sulfoxide, glycerol formal and sodium chloride. Conveniently, the non-toxic and non-irritant liquid is an aqueous isotonic solution suitable for ophthalmic use. Conveniently, the porous polymer is a hydrophilic polymer having a contact angle with the non-toxic and non-irritant fluid of less than 90°, more conveniently less than 75°. Conveniently, the porous polymer is provided with pores basically having the Gaussian distribution. Conveniently, the size of the pores grows from inside towards the surface of the functional part. Conveniently, positive capillary pressure is established in the pores of the porous polymer upon a contact with the non-toxic and non-irritant fluid. Conveniently, the porous polymer is selected from a set comprising polyurethane, polyurea, polyvinyl alcohol and its derivatives, and the derivatives of polyacrylate and polymethacrylate acid. Conveniently, the porous polymer has a covalent cross-linkage. Conveniently, the derivatives of polyacrylate and polymethacrylate acid include amides, nitriles and esters with polyols. Conveniently, said esters are created by 2-hydroxyethyl methacrylate. Conveniently, the functional part has a rounded application end for a non-aggressive contact with the area around the eye. Conveniently, the functional part has a cylindrical shape of a diameter 3 to 8 mm, more conveniently 4 to 6 mm, which conically passes at its end into a rounded application end having a diameter 0.5 mm to 3 mm, more conveniently 1 to 2 mm. Conveniently, at the moment of the non-aggressive contact with the area around the eye the non-toxic and non-irritant fluid is contained in a volume of 20 to 95. %, more conveniently 33 to 67% of the maximum retention capacity of the functional porous polymer part. Conveniently, the packaging is closed by a watertight cap that is: connected at one end to the functional part and at the other end to the handle.

### BRIEF DESCRIPTION OF THE DRAWINGS

On the attached drawings:

FIG. 1 schematically shows an example of the design of the device, containing the functional part 1 with the applicator end 1A provided with a concave recess 1B and a concave bulge 1C, and with the handle 2;

FIGS. 2A and 2B show two examples of connecting the functional part 1 of the device with the handle 2 in a longitudinal and a transverse cross-section, while the marking of these parts is identical to that in FIG. 1;

FIG. 3 shows a longitudinal and a transversal cross-section of a advantageous shape of the functional part 1 of the device;

FIG. 4 shows the convenient design of the device with its functional part 1 sealed in a watertight packaging 3, sealed with a watertight cap 4, the handle 2 passing through it in a waterproof way, wherein the handle 2 and the cap 4 make up conveniently a single integral part from the same material;

FIG. 5 shows an example of the device design with a disposable functional part 1 and with a handle 2 for repeated use, whereas the functional part 1 is closed with a watertight cap 4 in the packaging 3, the cap being integrated with the inner part 2A of the handle 2 and provided with a nut part 5A, the handle 2 being provided with a corresponding base part 5B; and

FIG. 6 shows an example of the design of the device consisting only of the functional part with the applicator member 1A containing a concave recess 1B and a concave bulge 1C.

### EXAMPLES OF THE EMBODIMENT OF THE INVENTION

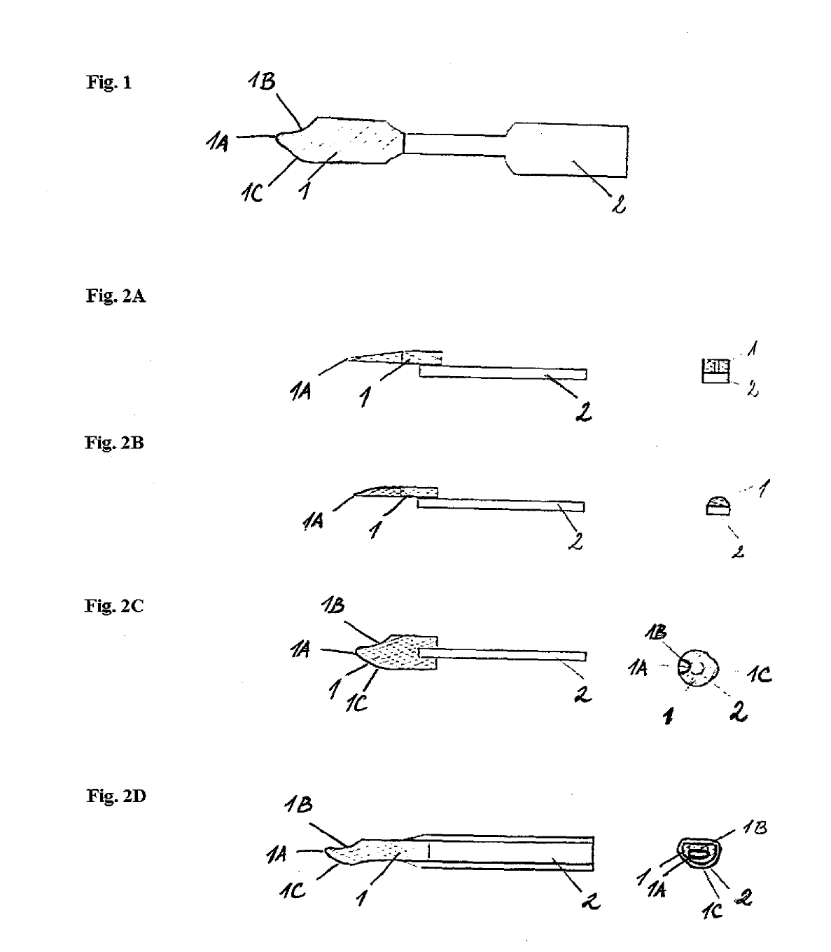

Direct application of contact lenses by means of fingers is a great shortage of the application of contact lenses. It is substantially reduced or overcome by using the device for a manipulation with contact lenses within the eye area in accordance to this invention, the device using for the manipulation with the contact lenses an appropriately shaped soft porous hydrophilic polymer, advantageously a hydrogel or a silicone-hydrogel. The device contains as a major and substantial part appropriately, with regard to its purpose, shaped part from a soft porous polymer containing at least in the moment of the contact with the contact lens and the eye area a non-toxical and non-irritating aquous fluid. The device consists at least of a substantial functional part 1, schematically showed on FIG. 1. The functional part 1 is formed by a shaped soft porous part from a hydrophilic polymer, which is prior to the manipulation with the contact lens and the application within the eye area, at least partly soaked with a physiologically unobjectionable and non-irritant fluid. The applicator end 1A is intended for a direct contact with the contact lens or with the eye surface or its neighbourhood area.

The device further conveniently includes a handle 2 that is used for a manipulation required for fixing the contact lens, its insertion into the eye area, as well as for a possible shifting it on the eye, and for the removal of the contact lens from the eye area. The handle 2 is at the same time a carrier of the functional part 1 and it conveniently forms a single integral part with it. The handle 2 is usually made of a suitable plastic material but it may also comprise other materials, such as metals or wood.

The porous functional part 1 has a shape optimised for the contact with the contact lens, its removal from the case and for the subsequent transfer of the contact lens into the eye area and for placing it onto the eye. In case of an oval or a circular profile, the handle 2 may be positioned either inside the functional part 1 as shown in FIG. 2A, or the functional part 1 may be positioned inside the handle 2, as shown in FIG. 2B.

The functional part 1 and the handle 2 may be permanently connected in various ways, e.g. by gluing, welding, casting the functional part 1 around a suitably shaped handle 2 and the like. The connection may also be temporary in a suitable manner, e.g. by means of threads, a bayonet or a Luer lock or in a similar way.

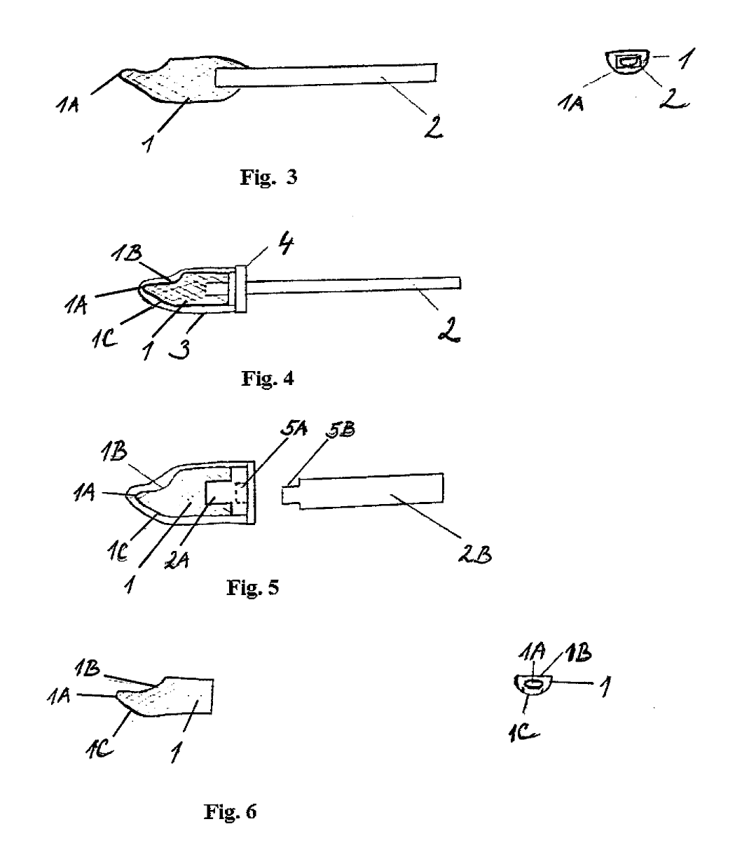

The applicator end 1A of the functional part 1 is conveniently rounded, e.g. being hemispherical, and the cross-section of the functional part 1 is conveniently tapered from the handle 2 to the applicator end 1A with a smaller rounded area on one side, as schematically shown in FIG. 3.

The length of the functional part 1 is advantageously approximately between 3 mm and 30 mm, most conveniently between 5 and 20 mm. The transverse dimension, e.g. the maximum diameter, of the functional part 1 is conveniently between 2 and 10 mm, best between 4 and 8 mm. The length of the handle 2 may be approximately from 10 mm to 100 mm, conveniently between 30 and 65 mm.

The material of the functional part 1 has to be sufficiently soft, conveniently shaped and deformable with a use of a minimum force, in order to allow expulsion of part of the fluid from the pores and during the reverse sucking the fluid to adhere with the contact lens without damaging it, both during the removal of the contact lens from the case and during the application of the contact lens within the eye area. For this reason, the volume of the pores should be more than 20%, and conveniently more than 30% of the hydrated volume of the functional part. The porosity should be between 15% and 98%, conveniently between 25% and 90% of the volume.

The size of the pores is important too, the average diameter of which should be between approximately 0.1 to 500 micrometers, conveniently from approx. 1 to 200 micrometers. For the size of the pores it is convenient not to be uniform, but having a certain distribution of the diameters corresponding, for instance, to a Gaussian distribution. It is also possible to distribute the pores from inside outwards toward the surface, advantageously in such a way, the size of the pores also increases from the interface with handle 2 toward the surface of the functional part 1.

The wettability of the pore surfaces is also important, so that a positive capillary pressure is preferentially created in the pores. To this purpose, it is convenient to select polymers, the surface of which has a wetting angle to the physiologically unobjectionable fluid contained in the pores at a room temperature of less than 90°, conveniently less than 75°. Hydrogels are preferably convenient, i.e. polymers insoluble in water, but absorbing water, i.e. swelling in the presence of water, up to a certain volume. Hydrogels may be covalently or physically cross-linked polymers of various chemical compositions. Low to medium hydrophilic hydrogels with balanced water content from 20% weight to 60% weight, preferably between 30% and 50% weight, are especially convenient for the purpose of the invention. Said hydrogels being tolerant also to fine fabrics and capable of surface absorption of impurities of both hydrophilic and hydrophobic character, said hydrogels being also capable of holding water fluid in their pores, which may be expelled by relatively small mechanical pressure. This pressure may be controlled by the size and by the hydrophilic character of the pores. For the application, the porous polymer functional part 1 should be at least partially moistened with a physiologically unobjectionable fluid.

The main condition for the selection of polymers for the functional part 1 is their health harmlessness certification, especially the fact that they do not contain toxic or irritant substances, or cannot emit such substances during the storage and use. Experts may draft many polymers and copolymers that fulfil the functional conditions of the invention. Thus, for instance, polyurethanes and polyureas, including their resultant copolymers with polyols can be used. Another preferred group of polymers comprises vinyl polymers and copolymers, e.g. polymers and copolymers of polyvinyl alcohol or vinyl acetate. A further preferred group comprises polymers and copolymers of acrylates and methacrylates, such as polymeric amides and esters of acrylate and methacrylate acids. An example is e.g. a sparsely cross-linked copolymer 2-hydroxyethyl methacrylate (HEMA) with small quantity of bis-ethylene glycol dimethacrylate. Polymers containing iogenous groups, such as carboxyls, sulfonamides, tertiary amines, etc. are also convenient. Some of the possible compositions shall be stated in the Examples. The fluid contained in the pores is a non-toxic and non-irritant fluid, conveniently an aqueous solution of salts or of water miscible organic non-toxic compounds. Examples of such organic compounds are, for instance, compounds with two or more hydroxyl groups, such as, sugars, glycerine, 1,2 propylene glycol, triethylene glycol, etc. Other suitable compounds may be dimethyl sulfoxide or glycerol formal. Such compounds may be present in the solution up to an approximate concentration of 80% weight, but usually their presence in a smaller volume is also sufficient. Isotonic solutions are mainly convenient, for instance, of suitable salts, such as NaCl. This solution can further contain suitable antiseptics or other active substances, commonly used in ophthalmic formulations, e.g. in so-called boric acid. It is important that they are non-irritant to the eye and are adequately stable during storage and use of the device. It is suitable for the application, that the overall fluid volume present in the functional part is adequately smaller than its maximum retention capacity for the given fluid. The retention capacity of the porous hydrogel is to be understood as the overall content of the fluid retained in the structure of the hydrogel as well as the fluid retained in fully-filled pores. When the fluid content is too high, the fluid could be expelled from the pores during application and could flow from around the eye, smear the make-up, etc. If it is too low, e.g. substantially lower than the balanced water content in the hydrogel, the surface of the functional part could be then too rough and it can damage or irritate the surface of the lens, eye, edges of the eyelid, etc. According to our experience, at the moment of the contact with the eye area said fluid should be contained in a volume equivalent to 20% up to 95%, conveniently 33% to 67% of the maximum retention capacity of the functional part from the porous polymer. The fluid content may be adjusted just before the application e.g. by wringing out the excess fluid, or on the contrary by soaking of a dry device in the fluid. Nevertheless, more convenient it is to store and to supply the device suitably packed with an optimum fluid volume.

For this reason, apart from the functional part 1 and the handle 2 the device is conveniently provided with a watertight packaging 3 that protects the functional part 1 from drying and contamination. The packaging 3 may contain more than one device and these can be taken out gradually for the usage. Such packaging may be, for instance, a bottle with a wide neck and screw cap, or a plastic casing, etc. Nevertheless, individual packaging for each device is convenient, providing more comfortable usage and guaranting better the device is clean. Such an individual packaging 3 may consist, for instance, of a plastic “blister” covered with removable foil or packaging for the entire device from welded plastic foil. Especially convenient is the packaging 3 shaped from a plastic material in accordance with the shape of the functional part 1, as shown in FIG. 4. The packaging 3 is conveniently sealed with a cap 4 attached to the handle 2, eventually forming one integral part together.

The entire device may be sealed in an impermeable packaging protecting it from a contamination and retaining said aqueous, physiologically unobjectionable fluid. In a disposable design, this packaging could be made, for instance, of a steam-proof plastic foil, which can be torn to remove the device. The plastic films being especially suitable to be used are, for instance, from polyethylene, polyethylene terephthalate (PET) or from metallized plastic foil. The handle 2 may be provided with an end designed to facilitate the tearing of the packaging.

As another alternative there is a divided packaging containing the device, advantageously a two-piece packaging from a suitable plastic material, such as polypropylene (PP), where both pieces are made by an injection moulding and being mutually connected by a watertight dismountable joint, such as a screw joint, Luer lock or bayonet lock. Such packaging may be used both for a one-time and for a repeated usage of the device, possibly also for its hygienic storage before being disposed as a waste.

Another advantageous arrangement with the handle 2 consisting of two parts is schematically shown on FIG. 5. The plastic packaging 3 has a watertight cap 4 and contains the functional part 1 and one part 2A of the device handle 2. The other part 2B of the device handle 2 is not permanently connected to the part 2A, but it is connected just before the use, after opening the cap 4. The cap 4 and the part 2A conveniently constitute a single component. After the use, the part 2B is disconnected from the part 2A, which is together with the connected functional part 1 and with the packaging 3 disposed as a waste, while the part 2B of the device handle 2 is kept for a further use. The parts 2A and 2B are provided with a suitable system for quick coupling and release, such as a bayonet lock, a Luer Lock, a lock being common in automatic pencils, etc., schematically shown in FIG. 5 as parts 5A and 5B.

A convenient method for a production of the functional part 1 is casting of a liquid precursor in a mould under conditions suitable for creation of a porous polymer. Such processes are well-known to experts in the plastics branch. The casting mould is conveniently formed by the packaging 3. The composition of the liquid precursor is conveniently selected such, that the result of its hardening is non-toxic and non-irritant, not requiring to be extensively washed or cleaned in a different way. This eliminates various production operations and reduces production costs. Moreover, the reduction of the number of operations also reduces the possibility of contamination. A convenient material for the functional part 1 is a porous hydrogel. Creation of a spongy gel structure can be attained, for instance, by using a precursor that contains more water fluid than the created hydrogel can retain by its balanced swelling. In such a case, so-called, “syneresis” occurs, whereby the polymer and fluid mutually exude each other resulting in a creation of a spongy structure. This effect is well-known to polymer chemists, who can find number of actual systems that fulfil such condition. An example of the creation of a porous hydrogel by means of a physical process may be, for instance, recurrent freezing and thawing of polyvinyl alcohol solutions, which by freezing and subsequent thawing create a fine spongy gel structure, whose consistency, porosity, etc. can be controlled, e.g. by a concentration of the polymer in the solution, by a temperature and by number of cycles. Another example may be the co-polymerisation of HEMA with a suitable cross-linker in adequately diluted aqueous solutions. The initiation of polymerisation using radical initiators is suitable, for instance, organic or inorganic peroxides or Azo-compounds. Initiators are decomposed by heat, UV radiation or reaction with suitable reducing compounds, so-called, redox initiation systems. Such water-soluble initiators whose reaction products are unobjectionable in terms of health are especially convenient and the products do not need to be removed from the reaction carefully. An example may be hydrogen peroxide or Ammonium persulfate.

The hardening of the liquid precursor is conveniently done in a presence of the handle 2 in the mold, which is thus installed into the hydrogel functional part 1 without a need for additional operations. However, it is alternatively possible to create a hole or depression in the functional part 1 as a receptor for the handle 2, and to bond the handle 2 into this receptor using a suitable adhesive, e.g. cyanoacrylate glue.

The basic function of the device is a contactless manipulation with the contact lenses and it can be described in a following way:

a) The device is removed from its packaging and the case of the contact lens is opened. The contact lens is moved by the application end 1A to the periphery of the case. The contact lens is taken from beneath and it is placed onto the concave recesses 1B or onto the convex bulge 1C.

b) The contact lens sticks on the porous polymer without being damaged.

c) The device is moved in the eye area where the contact lens is placed through a non-aggressive touch onto the eye cornea.

d) When a manipulation with the contact lens on the eye is necessary, it is possible to move it by the applicator end 1A within the eye area.

e) In order to remove the contact lens from the eye, the applicator end 1A is used again to do it, thanks to the applicator end 1A the contact lens is non-agressively moved to the inner corner of the eye.

f) After the contact lens is moved to the inner corner of the eye it is possible to easily take it from beneath by the part 1 and the contact lens sticks again on the porous polymer.

g) The device with the contact lens is removed from the eye area.

After its removal from the eye area, it is possible to clean the device, wet it with a fresh aqueous fluid and to use it again. In terms of the user's hygiene, safety and comfort it is, however, advantageous to discard the device after the first use.

### Example 1

The polyethylene container with a volume of 1.5 ml with cap is used primarily as a mini test-tube or sample container for small samples (FISCHER SCIENTIFIC, CATALOGUE N^{o} 2103.3502); it is used as a casting mould as well as a watertight packaging 3 (see FIG. 4 and FIG. 5) for the hydrogel functional part 1. The mould is modified by cutting off the cap, which is then replaced by the polyethylene part that is used as an anchor for the part 2A, the cap 4 and the connecting part 5A with a bayonet lock according to FIG. 5.

The mould is filled with a 10% PVA and 0.8% NaCl water solution, closed from above with said polyethylene part and subjected to 3 cycles of freezing to −20° C. and thawing to +20° C. The solution is prepared by dissolving PVA of a molecular weight 80 k Dalton and an hydrolysis level above 99% in an isotonic solution of NaCl at a temperature above 95° C. By recurrent freezing and thawing, the polymer is exuded in an insoluble form from the water and the porous hydrogel with open pores is thus created, from which it is possible to easily wring excess water by an application of a small pressure.

The product is stored in this form. Before its use, a metallic handle 2 is attached to the cap according to FIG. 5; the handle 2 having the second part 5B of the bayonet lock at its end, and the porous hydrogel functional part 1 is pulled out from the packaging 3 and used in the eye area. After this, the handle 2 is disengaged and kept for a further use, while the used functional part 1 is returned to the packaging 3 and discarded as a waste.

### Example 2

The polyethylene mould from the example 1 is partly filled with a de-aerated solution of 30% by weight of HEMA in a 1% aqueous solution of ammonium sulphate. HEMA contains 0.25% by weight of ethylene glycol dimethacrylate and 5% by weight of triethylene glycol dimethacrylate. The filled mould is closed then with the rubber cap 4, through which a polypropylene handle 2 passes tightly as seen on FIG. 4. The closed moulds are heated in a water bath to 80° C. over a period of 3 hours, being cooled thereafter and stored in a plastic container. Thanks to the thermal polymerisation, a hydrogel sponge constituting the functional part 1 according to FIG. 4 is created in the mould formed by the packaging 3. Before its use the hydrogel functional part 1 is removed from the packaging 3 using the handle 2 and the rounded applicator end 1A of the functional part 1 is used for the manipulation with the contact lens. After the manipulation with the contact lens the functional part 1 including the handle 2 and the cap is returned into the packaging 3, which is disposed of as waste at the proper moment.

### Example 3

The procedure from example 2 is repeated up to the end of the thermal polymerisation. Then, the functional part 1 with cap 4 and the handle 2 from FIG. 4 is removed from the mould; the cap 4 is removed and recycled for further use. The functional part 1 with the attached handle 2 are washed then in distilled water and soaked in a 30% glycerine water solution. After 24 hours, the excess solution is drained by spinning and the parts are dried in the air for 12 hours. The water partly evaporates from the functional part 1 and a concentrated glycerine is retained in it, serving as a softener and partly also as a hydrogel preservative. The finished product is sealed in a polyethylene foil.

Before its use the foil is torn, the product is taken out and used for the manipulation with the contact lens into the eye area either directly, or after moistening with an ophthalmic solution. When using the device for the manipulation with the contact lens the device according to the example is removed from the plastic bag a possibly it is olse soaked with an ophthalmic solution.

1. A device for a manipulation with a contact lens with an eye area, characterized in that it consists of a functional part (1) from a porous polymer, especially from a hydrophilic polymer, which is adapted by its shape for a non-aggressive contact with an eye area and by its porousness for absorbing a non-toxic and non-irritant fluid, wherein in the surface of the functional part (1) at least one concave recess (1B) and/or from the surface of the functional part (1) extending at least one convex bulge (1B) is provided on a front applicator end (1A) of the functional part (1).

2. The device according to claim 1, characterized in that it includes a handle (2) permanently connected with or separable from the functional part (1).

3. The device according to claim 1, characterized in that it is sealed in a watertight packaging (3).

4. The device according to claim 1, characterized in that the porous polymer of the functional part (1) has an average porosity of 20 to 98% by volume, related to the total volume of the porous polymer.

5. The device according to claim 1, characterized in that the porous polymer of the functional part (1) has an average pore size of 0.1 to 250 micrometers.

6. The device according to claim 1, characterized in that the porous polymer of the functional part (1) is a deformable porous polymer.

7. A method of manufacturing the device according to claim 1, characterized in that the functional part (1) is obtained from an aqueous solution of a porous polymer precursor by separating out the porous polymer by means of removal of water from said solution, possibly followed by connecting the acquired functional part (1) with the handle (2), and the functional part (1) alone or together with the handle (2) is eventually sealed in a watertight packaging (3).

8. The method according to claim 7, characterized in that the aqueous solution of the porous polymer precursor is an aqueous solution of polyvinyl alcohol and the porous polymer is separated out from this solution by removing out the water by means of a recurrent freezing and thawing of said solution.

9. A method of manufacturing the device according to claim 1, characterized in that the aqueous solution of the porous polymer precursor is an aqueous solution containing a major part of 2-hydroxyethyl methacrylate and minor part of a multifunctional cross-linker and the separation of the porous polymer is done by a radical polymerisation in the presence of a water soluble initiator of the radical polymerisation, eventually followed by connecting the acquired functional part (1) with the handle (2), and the functional part (1) alone or together with the handle (2) is eventually sealed into a watertight packaging (3).

10. The method according to claim 7, characterized in that the separation of the porous polymer from the aqueous solution of the porous polymer precursor is carried out directly in the watertight packaging (3).
